SEQUENCE LISTING (3 sequences)

SEQ ID NO: 1 - protein, 422 aa, Micrococcus candicans ATCC 8456
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 2 - protein, 422 aa, Pseudomonas putida
MNANDNVVIVGTGLAGVEVAFGLRASGWEGNIRLVGDATVIPHHLPPLSKAYLAGKATAESLYLRTPDAYAAQNIQLLGGTQVTAINRDRQQVILSDGRALDYDRLVLATGGRPRPLPVASGAVGKANNFRYLRTLEDAECIRRQLIADNRLVVIGGGYIGLEVAATAIKANMHVTLLDTAARVLERVTAPPVSAFYEHLHREAGVDIRTGTQVCGFEMSTDQQKVTAVLCEDGTRLPADLVIAGIGLIPNCELASAAGLQVDNGIVINEHMQTSDPLIMAVGDCARFHSQLYDRWVRIESVPNALEQARKIAAILCGKVPRDEAAPWFWSDQYEIGLKMVGLSEGYDRIIVRGSLAQPDFSVFYLQGDRVLAVDTVNRPVEFNQSKQIITDRLPVEPNLLGDESVPLKEIIAAAKAELSSA

SEQ ID NO: 3 - protein, 107 aa, Pseudomonas putida
MSKVVYVSHDGTRRELDVADGVSLMQAAVSNGIYDIVGDCGGSASCATCHVYVNEAFTDKVPAANEREIGMLECVTAELKPNSRLCCQIIMTPELDGIVVDVPDRQW

(19) United States
(12) Patent Application Publication
(10) Pub. No.: US 2019/0127769 A1
(43) Pub. Date: May 2, 2019

(54) UNSATURATED AMINO ACIDS

(71) Applicant: EVONIK DEGUSSA GMBH, Essen (DE)

(72) Inventors: Thomas HAAS, Münster (DE); Anja HECKER, Münster (DE); Thomas BÜLTER, Duisburg (DE); Wolfgang KROUTIL, Graz (AT); Kurt FABER, Graz (AT)

(73) Assignee: EVONIK DEGUSSA GMBH, Essen (DE)

(21) Appl. No.: 16/094,334

(22) PCT Filed: May 3, 2017
(86) PCT No.: PCT/EP2017/060552

§ 371 Date: Oct. 17, 2018

(30) Foreign Application Priority Data
May 4, 2016 (EP) 16168227.3

(51) Int. Cl. C12P 13/04; C12N 9/08; C12N 9/02; C07K 14/33; C07K 14/21; C07C 319/18

(52) U.S. Cl. CPC C12P 13/04; C12Y 111/02004; C12N 9/0065; C12N 9/0095; C12Y 118/01002; C07K 14/33; C12Y 102/01002; C07K 14/21; C07C 319/18; C12N 9/0071; C12Y 114/14001; C12N 9/0008; C12Y 118/01005

(57) ABSTRACT

There is provided a method of producing at least one unsaturated amino acid from at least one amino acid comprising at least two carbonyl groups, the method comprising (a) contacting a recombinant microbial cell with a medium comprising the amino acid comprising the carbonyl groups, wherein the cell is genetically modified to comprise—at least a first genetic mutation that increases the expression relative to the wild type cell of an enzyme (E) selected from the CYP152 10 peroxygenase family, and—at least a second genetic mutation that increases the expression relative to the wild type cell of at least one NAD(P)+ oxidoreductase (E2) and the corresponding mediator protein.

### FIELD OF THE INVENTION

The present invention relates to a biotechnological method that is capable of producing at least one unsaturated amino acid from at least one amino acid, wherein the starting amino acid has at least two carbonyl groups. In particular, the resultant unsaturated amino acid has at least one terminal double carbon bond.

### BACKGROUND OF THE INVENTION

Amino acids with an unsaturated side chain has several new uses. In particular, these amino acids may be used as building blocks for other useful compounds. For example, these alkene moieties can be used in bioorthogonal synthesis strategies to form hybrid structures, introduce chemical probes into biomolecules, or link large fragments with each other.

One of the more important and useful unsaturated amino acids is a vinylglycine (2-aminobut-3-enoic acid). Vinylglycine, is a natural, non-protein a-amino acid and is usually isolated from fungi and is known to irreversibly inhibit many enzymes that use pyridoxal phosphate (PLP) as a cofactor. Vinylglycine and derivatives thereof have thus been utilized as enzyme inhibitors and/or antibiotics.

A three-step synthesis of vinylglycine has been developed using but-3-enenitrile as the starting material based on the Neber rearrangement of the corresponding N-chloroimidate. However, this method is very complicated and the starting material difficult to access. Other more common ways of preparing L-vinylglycine includes the pyrolysis of protected methionine sulfoxide (MetO) and thermolysis of aryl selonoxides obtained from either L-glutamate, L-homoserine, or L-homoserine lactone. However, due to the high vacuum (≤3 mm Hg) and temperature (>150° C.) requirements, isomerization is a consistent problem for the reaction. Further, the chances of the L-vinylglycine converting to the thermally stable β-methyldehydroalanine is also very high in these methods. This reduces the yield of L-vinylglycine. It is also difficult to isolate vinylglycines from the resultant reaction mixture by chromatography using this method. Vinylglycines may also be produced by contacting butadiene with an epoxidase to produce butadiene epoxide which is then hydrolysed, where the epoxide group is converted to the diol. The diol is then oxidised to the hydroxy acid and aminated to form vinylglycine. However, this method of forming vinylglycine requires many steps and is therefore costly, and may result in loss of products along the way.

There is thus a need in the art to find a different means of producing unsaturated amino acids including vinylglycine that does not use a non-pyrolytic large scale approach and that uses an easily available starting material. In particular, there is a need to develop a biotechnological production process for unsaturated amino acids using an easily available and reasonably priced raw material.

### DESCRIPTION OF THE INVENTION

The present invention attempts to solve the problems above by providing a biotechnological means of producing at least one unsaturated amino acid from at least one amino acid with at least two carbonyl groups. In particular, there is provided a genetically modified cell with a specific enzyme cascade for the biocatalytic synthesis of a terminal alkenyl group by oxidative decarboxylation of the amino acid with the two carbonyl groups. The cell does not require H₂O₂ for this step of decarboxylation. The enzyme cascade comprises a decarboxylation reaction which is H₂O₂-independent and may be catalysed by at least one P450 monooxygenase. In particular, the cell expresses an enzyme, for example OleT, which may be capable of optimising a biocatalytic system to produce at least one alkenyl group from a carboxyl group in an amino acid using decarboxylation reactions.

According to one aspect of the present invention, there is provided a method of producing at least one unsaturated amino acid from at least one amino acid comprising at least two carbonyl groups, the method comprising

(a) contacting a recombinant microbial cell with a medium comprising the amino acid comprising the carbonyl groups,

wherein the cell is genetically modified to comprise
at least a first genetic mutation that increases the expression relative to the wild type cell of an enzyme (E₁) selected from the CYP152 peroxygenase family, and
at least a second genetic mutation that increases the expression relative to the wild type cell of at least one NAD(P)+ oxidoreductase (E₂) and the corresponding mediator protein.

In contrast to the usual chemo-catalytic routes which are usually used to produce alkenyl groups, the method according to any aspect of the present invention may use whole cells or isolated enzymes. This allows for the method to be carried out under mild reaction conditions, thereby enabling sustainable processes with minimal waste emission. This is an unexpected result as prior art (Fujishiro T., 2007 and Matsunaga I., 2002) reported that P450 reductase systems such as ferredoxin and ferredoxin reductase did not support the activity of P450_{BSβ} and P450_{SPα}.

Further, the method according to any aspect of the present invention allows for large scale production of unsaturated amino acids from the amino acids with the carbonyl groups that are used as substrates.

The method according to any aspect of the present invention has further advantages such as it uses O₂ as an oxidant, that makes the process more efficient than the methods known in the art which use H₂O as an oxidant; the method allows for electron transfer from renewable resources and the method according to any aspect of the present invention also results in significantly high production of unsaturated amino acids.

The amino acid comprising at least two carbonyl groups according to any aspect of the present invention may be selected from the group consisting of aspartic acid, glutamic acid, asparagine and glutamine. These amino acids comprise at least two carbonyl (C═O) groups. One of the carbonyl groups is part of the carboxyl group that forms the backbone of an amino acid, the other, may be available to form an alkenyl group according to any aspect of the present invention.

In one example, the amino acid comprising at least two carbonyl groups according to any aspect of the present invention may be glutamic acid and/or derivatives thereof. Derivatives of glutamic acid include esters and/or amides of glutamic acid. In particular, derivatives of glutamic acid may include alkoxy esters, N-Boc protected derivatives, N-Acetyl protected derivatives, salts of glutamic acid, such as sodium glutamate etc., and homo or hetero peptides of glutamic acid. In a further example, the amino acid comprising at least two carbonyl groups according to any aspect of the present invention may be N-acetylglutamate.

In yet another example, a mixture of glutamic acid and at least one derivative of glutamic acid may be used as a substrate according to any aspect of the present invention for producing vinylglycine and/or the respective derivative. The derivative of vinylglycine formed may be dependent on the derivative of glutamic acid used as the substrate.

Unsaturated amino acids may be any amino acid with at least one alkenyl group. In particular, the unsaturated amino acid may comprise at least one carboxyl, amino and alkenyl group. Examples of unsaturated amino acids may be selected from the group consisting of vinylglycine, dehydroalanine, β-methyldehydroalanine and the like.

In particular, the unsaturated amino acid may be vinylglycine and/or derivatives thereof. Vinylglycine has a general chemical formula of C₄H₇NO₂ and a structural formula of:

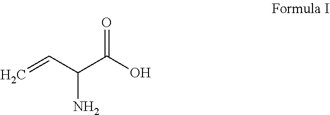

The derivatives of vinylglycine may be selected from the group consisting of amides of vinylglycine, esters of vinylglycine, rhizobitoxin, aminoethoxyvinylglycine, amine esters of vinylglycine, amide esters of vinylglycine, HCl-Salts of vinylglycine, a protected amino acid of vinylglycine and the like. Protection groups might be Boc, Fmoc, Cbz or ester moieties or a combination of them. In particular, the derivatives of vinylglycine may be selected from the group consisting of rhizobitoxin, aminoethoxyvinylglycine, amine esters of vinylglycine, amide esters of vinylglycine, amides of vinylglycine, esters of vinylglycine and peptides of vinylglycine. In one example, the derivative of vinylglycine may be N-acetylvinylglycine.

The cell according to any aspect of the present invention may refer to a wide range of microbial cells. In particular, the cell may be a prokaryotic or a lower eukaryotic cell selected from the group consisting of Pseudomonas, Corynebacterium, Bacillus and Escherichia. In one example, the cell may be Escherichia coli. In another example, the cell may be a lower eukaryote, such as a fungus from the group comprising Saccharomyces, Candida, Pichia, Schizosaccharomyces and Yarrowia, particularly, Saccharomyces cerevisiae. The cell may be an isolated cell, in other words a pure culture of a single strain, or may comprise a mixture of at least two strains. Biotechnologically relevant cells are commercially available, for example from the American Type Culture Collection (ATCC) or the German Collection of Microorganisms and Cell Cultures (DSMZ). Particles for keeping and modifying cells are available from the prior art, for example Sambrook/Fritsch/Maniatis (1989).

The phrase “wild type” as used herein in conjunction with a cell or microorganism may denote a cell with a genome make-up that is in a form as seen naturally in the wild. The term may be applicable for both the whole cell and for individual genes. The term ‘wild type’ may thus also include cells which have been genetically modified in other aspects (i.e. with regard to one or more genes) but not in relation to the genes of interest. The term “wild type” therefore does not include such cells or such genes where the gene sequences have been altered at least partially by man using recombinant methods. A wild type cell according to any aspect of the present invention thus refers to a cell that has no genetic mutation with respect to the whole genome and/or a particular gene. Therefore, in one example, a wild type cell with respect to enzyme E₁ may refer to a cell that has the natural/non-altered expression of the enzyme E₁ in the cell. The wild type cell with respect to enzyme E₂, E₃, etc. may be interpreted the same way and may refer to a cell that has the natural/non-altered expression of the enzyme E₂, E₃, etc. respectively in the cell.

Any of the enzymes used according to any aspect of the present invention, may be an isolated enzyme. In particular, the enzymes used according to any aspect of the present invention may be used in an active state and in the presence of all cofactors, substrates, auxiliary and/or activating polypeptides or factors essential for its activity. The term “isolated”, as used herein, means that the enzyme of interest is enriched compared to the cell in which it occurs naturally.

The enzyme may be enriched by SDS polyacrylamide electrophoresis and/or activity assays. For example, the enzyme of interest may constitute more than 5, 10, 20, 50, 75, 80, 85, 90, 95 or 99 percent of all the polypeptides present in the preparation as judged by visual inspection of a polyacrylamide gel following staining with Coomassie blue dye.

The cell and/or enzyme used according to any aspect of the present invention may be recombinant. The term “recombinant” as used herein, refers to a molecule or is encoded by such a molecule, particularly a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule.

Whether or not a nucleic acid molecule, polypeptide, more specifically an enzyme used according to any aspect of the present invention, is recombinant or not does not necessarily have implications for the level of its expression. However, in one example one or more recombinant nucleic acid molecules, polypeptides or enzymes used according to any aspect of the present invention may be overexpressed. The term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell. The person skilled in the art is familiar with numerous ways to bring about overexpression. For example, the nucleic acid molecule to be overexpressed or encoding the polypeptide or enzyme to be overexpressed may be placed under the control of a strong inducible promoter such as the lac promoter. The state of the art describes standard plasmids that may be used for this purpose, for example the pET system of vectors exemplified by pET-3a (commercially available from Novagen). Whether or not a nucleic acid or polypeptide is overexpressed may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions. Thus, in various examples of the present invention, to function more efficiently, a microorganism may comprise one or more gene deletions. Gene deletions may be accomplished by mutational gene deletion approaches, and/or starting with a mutant strain having reduced or no expression of one or more of these enzymes, and/or other methods known to those skilled in the art. In one example, the cell according to any aspect of the present invention may be genetically modified to comprise at least a first genetic mutation that increases the expression relative to the wild type cell of an enzyme (E₁) selected from the CYP152 peroxygenase family. In this example, the enzyme E₁ may be overexpressed in a wild type cell where the expression of enzyme E₁ may be absent or expressed at the wild type level. Similarly, in the same example or in another example, the enzyme, NAD(P)+ oxidoreductase (E₂) and the corresponding mediator protein may be overexpressed relative to the expression of these enzymes and/or proteins in the wild type cell.

The enzyme (E₁) selected from the CYP152 peroxygenase family used according to any aspect of the present invention may be part of the superfamily of cytochrome P450 enzymes (CYPs) (Malca et al., 2011). Typically, P450 enzymes employ one or more redox partner proteins to transfer two electrons from NAD(P)H to the heme iron reactive center for dioxygen activation, and then insert one atom of O₂ into their substrates. The enzymes within the family of CYP152 peroxygenases have been identified to exclusively use H₂O₂ as the sole electron and oxygen donors. However, in the cell according to any aspect of the present invention, NAD(P)+ oxidoreductase (E₂) and the corresponding mediator protein may be used as the source of electron and oxygen donors. This is advantageous as in a large scale production of low-cost unsaturated amino acids with a terminal alkenyl group, the use of large amounts of peroxide is cost prohibitive, and high concentration of H₂O₂ can quickly deactivate biocatalysts. Accordingly, the use of NAD(P)+ oxidoreductase (E₂) and the corresponding mediator protein as a source of electrons provides a more cost-effective microbial production of unsaturated amino acids. This may be further explained in Liu et al., 2014.

In particular, enzyme E₁ may be selected from the group consisting of CYP_{SPα} (E₁ₐ), CYP_{BSB} (E_{1b}) (EC 1.11.2.4) and OleT (E_{1c}). More in particular, the enzyme E₁ may be OleT (E_{1c}) or a variant thereof. In one example, enzyme E₁ may comprise the sequence of ADW41779.1. In another example, the enzyme E₁ may have 50, 55, 60, 65, 70, 75, 80, 85, 90, 95, 98, 100% sequence identity to SEQ ID NO:1.

A skilled person would be capable of identifying the possible sequences of OleT that may be used to carry out the process of forming at least one unsaturated amino acid from at least one amino acid comprising at least two carbonyl groups. In one example, the skilled person may use the disclosure in Liu et al, 2014, Rude M. A, 2011, Schallmey, A., 2011, Fukada H., 1994, Belcher J., 2014 and the like to determine the structure and means of introducing OleT (E_{1c}) into a suitable cell and determining the expression of the enzyme in the cell. OleT (as compared to other H₂O₂-dependent enzymatic reactions) may lead to an artificial electron transfer system to result in higher yield.

The cell used in the method according to any aspect of the present invention may comprise a second genetic mutation that increases the expression relative to the wild type cell of at least one enzyme, the NAD(P)+ oxidoreductase (E₂) and the corresponding mediator protein. These enzymes belong to a family of oxidoreductases that oxidise the mediator protein and accept two electrons. In particular, NAD(P)+ oxidoreductases may use iron-sulphur proteins as electron donors and NAD⁺ or NADP⁺ as electron acceptors. Hannemann et al. discloses a list of various classes of redox-mediators that may be used as enzyme E₂ according to any aspect of the present invention. In one example, artificial/“chemical” redox mediators could transfer electrons either from reductases or electrical sources to the heme iron cluster.

More in particular, the NAD(P)+ oxidoreductase (EC 1.18.1.5) and the corresponding protein may be selected from the group consisting of:

(a) ferredoxin reductase (E₂ₐ) and ferredoxin; or

(b) putidaredoxin reductase (E_{2b}) and putidaredoxin (Schallmey, A., 2011).

In particular, E₂ may be CamA and the mediator protein may be CamB. E₂ may comprise 50, 55, 60, 65, 70, 75, 80, 85, 90, 95, 98, 100% sequence identity to SEQ ID: NO:2 and/or the mediator protein may comprise 50, 55, 60, 65, 70, 75, 80, 85, 90, 95, 98, 100% sequence identity to SEQ ID: NO:3.

In one example, in the cell according to any aspect of the present invention E₂ may be ferredoxin reductase (E₂ₐ) where ferredoxin may also be present and E₂ₐ may be capable of functionally interacting with E₁. In particular, the source of E₁ and E₂ may be the same or different. In one example, both E₁ and E₂ may come from the same source, for example from Alcanivorax borkumensis SK2 (accession number YP_691921). In this example, E₂ₐ and ferredoxin may have accession numbers YP_691923 and YP_691920, respectively.

In another example, in the cell used in the method according to any aspect of the present invention E₂ may be putidaredoxin reductase (E_{2b}) where putidaredoxin may also be present and E_{2b} may be capable of functionally interacting with E₁. In one example, E_{2b} may be from the P450_{cam} enzyme system from Pseudomonas putida. For putidaredoxin reductase, typically the amount of enzyme employed may be about 100 to 10,000 ca, 1000 to 5000 ca, 2000 to 4000 ca or in particular 3000 ca. The ca is the unit of activity of putidaredoxin reductase in mediating the oxidation of NADH by ferricyanide and is defined as 1 μmole of NADH oxidised per mg reductase per minute.

E₂ be a recombinant protein or a naturally occurring protein which has been purified or isolated. The E₂ may have been mutated to improve its performance such as to optimise the speed at which it carries out the electron transfer or its substrate specificity. The amount of reductase employed will depend on the exact nature of what is measured and the particular details of the assay but typically, the reductase will be present at a concentration of from 0 to 1000 μM, 0.001 to 100 μM, 0.01 to 50 μM, 0.1 to 25 μM, and in particular from 1 to 10 μM.

The cell used in the method according to any aspect of the present invention may further comprise at least a third genetic mutation that may increase the expression relative to the wild type cell of at least one enzyme (E₃) capable of cofactor regeneration. In particular, E₃ may be an enzyme capable of NAD(P)H regeneration. More in particular, E₃ may be a dehydrogenase/oxidoreductase which uses NAD(P) as electron acceptor (EC 1.1.1.X). Even more in particular, E₃ may be any enzyme with KEGG no. EC 1.1.1.X in the Brenda database as of 24 Feb. 2014. For example, E₃ may be selected from the group consisting of alcohol dehydrogenase, glycerol phosphate dehydrogenase, histidinol dehydrogenase, shikimate dehydrogenase, lactate dehydrogenase, 3-hydroxyaryl-CoA dehydrogenase, malate dehydrogenase, isocitrate dehydrogenase, glucose-6-phosphate dehydrogenase, formate dehydrogenase, horse liver alcohol dehydrogenase, glucose dehydrogenase, amino acid dehydrogenase, sorbitol dehydrogenase, 20-β-hydroxysteroid dehydrogenase and formaldehyde dehydrogenase. In particular, enzyme (E₃) may be selected from the group consisting of glucose dehydrogenase (E₃ₐ) (EC 1.1.99.10), phosphite dehydrogenase (E_{3b}) (EC 1.20.1.1) and formate dehydrogenase (E_{3c}) (EC 1.2.1.43) where glucose, phosphite and formate are used as reducing agents respectively. The presence of enzyme (E₃) in the cell used in the method according to any aspect of the present invention allows for cofactor regeneration that enables the process of producing unsaturated amino acids from amino acids with two carbonyl groups to be self-sustaining. No external energy would thus have to be introduced into the system of producing unsaturated amino acids. Accordingly, the cell according to any aspect of the present invention may be able to generate at least one unsaturated amino acid from an amino acid with at least two carbonyl groups in the presence of at least enzymes E₁, E₂ and/or E₃ without any external energy source needed.

In one example, the glucose dehydrogenase (E₃ₐ) may be NADP+-specific glucose dehydrogenase. The organism that serves as the source of glucose dehydrogenase (E₃ₐ) may not be subject to limitation, and may be a microorganism such as bacteria, fungi, and yeast. For example, a microorganism of the genus Bacillus, in particular Bacillus megaterium, may be the source. In another example, the source may be a microorganism belonging to the genus Cryptococcus, the genus Gluconobacter, or the genus Saccharomyces. In particular, a microorganism belonging to the genus Cryptococcus may be selected, more in particular, the microorganism may be selected from the group consisting of Cryptococcus albi dus, Cryptococcus humicolus, Cryptococus terreus, and Cryptococcus uniguttulatus.

In another example, enzyme E₃ may be phosphite dehydrogenase (E_{3b}) or formate dehydrogenase (E_{3c}). The organism that serves as the source of phosphite dehydrogenase (E_{3b}) or formate dehydrogenase (E_{3c}) may not be subject to limitation, and may be a microorganism such as bacteria, fungi, and yeast.

In one example, the cell according to any aspect of the present invention has increased expression relative to a wild type cell of enzymes E_{1c}, E₂ₐ and E₃ₐ. In another example, the cell according to any aspect of the present invention has increased expression relative to a wild type cell of E_{1c}, E₂ₐ and E_{3b}; E_{1c}, E₂ₐ and E_{3c}; E_{1c}, E_{2b} and E₃ₐ; E_{1c}, E_{2b} and E_{3b}; or E_{1c}, E_{2b} and E_{3c}.

The teachings of the present invention may not only be carried out using biological macromolecules having the exact amino acid or nucleic acid sequences referred to in this application explicitly, for example by name or accession number, or implicitly, but also using variants of such sequences. The term “variant”, as used herein, comprises amino acid or nucleic acid sequences, respectively, that are at least 70, 75, 80, 85, 90, 92, 94, 95, 96, 97, 98 or 99% identical to the reference amino acid or nucleic acid sequence, wherein preferably amino acids other than those essential for the function, for example the catalytic activity of a protein, or the fold or structure of a molecule may be deleted, substituted or replaced by insertions or essential amino acids are replaced in a conservative manner to the effect that the biological activity of the reference sequence or a molecule derived therefrom is preserved. The state of the art comprises algorithms that may be used to align two given nucleic acid or amino acid sequences and to calculate the degree of identity, see Arthur Lesk (2008), Thompson et al., 1994, and Katoh et al., 2005. The term “variant” is used synonymously and interchangeably with the term “homologue”. Such variants may be prepared by introducing deletions, insertions or substitutions in amino acid or nucleic acid sequences as well as fusions comprising such macromolecules or variants thereof. In one example, the term “variant”, with regard to amino acid sequence, comprises, in addition to the above sequence identity, amino acid sequences that comprise one or more conservative amino acid changes with respect to the respective reference or wild type sequence or comprises nucleic acid sequences encoding amino acid sequences that comprise one or more conservative amino acid changes. In one example, the term “variant” of an amino acid sequence or nucleic acid sequence comprises, in addition to the above degree of sequence identity, any active portion and/or fragment of the amino acid sequence or nucleic acid sequence, respectively, or any nucleic acid sequence encoding an active portion and/or fragment of an amino acid sequence. The term “active portion”, as used herein, refers to an amino acid sequence or a nucleic acid sequence, which is less than the full length amino acid sequence or codes for less than the full length amino acid sequence, respectively, wherein the amino acid sequence or the amino acid sequence encoded, respectively retains at least some of its essential biological activity. For example an active portion and/or fragment of a protease may be capable of hydrolysing peptide bonds in polypeptides. The phrase “retains at least some of its essential biological activity”, as used herein, means that the amino acid sequence in question has a biological activity exceeding and distinct from the background activity and the kinetic parameters characterising said activity, more specifically k_{cat} and K_{M}, are preferably within 3, 2, or 1 order of magnitude of the values displayed by the reference molecule with respect to a specific substrate. Similarly, the term “variant” of a nucleic acid comprises nucleic acids the complementary strand of which hybridises, preferably under stringent conditions, to the reference or wild type nucleic acid. A skilled person would be able to easily determine the enzymes E₁, E₂ and/or E₃ that will be capable of making unsaturated amino acids from amino acids with at least two carbonyl groups according to any aspect of the present invention.

An illustration of the difference in the reaction that takes place in the cell according to any aspect of the present invention in the presence of H₂O₂and the absence of H₂O₂ (i.e. in the presence of enzyme E₂ and the mediator protein instead) is shown in Scheme 1. In particular, in scheme 1(A), an enzymatic redox-cascade for decarboxylation of a carboxyl group to terminal-alkenyl groups is shown. The electrons are shown to be transferred from a hydride donor (e.g. glucose, formate or phosphite) via CamAB to OleT that catalyses the oxidative decarboxylation of carboxyl groups at the expense of atmospheric O₂ to terminal alkenyl groups. Side products detected are shown in brackets. In scheme 1(B), the same reaction in the presence of H₂O₂ is shown.

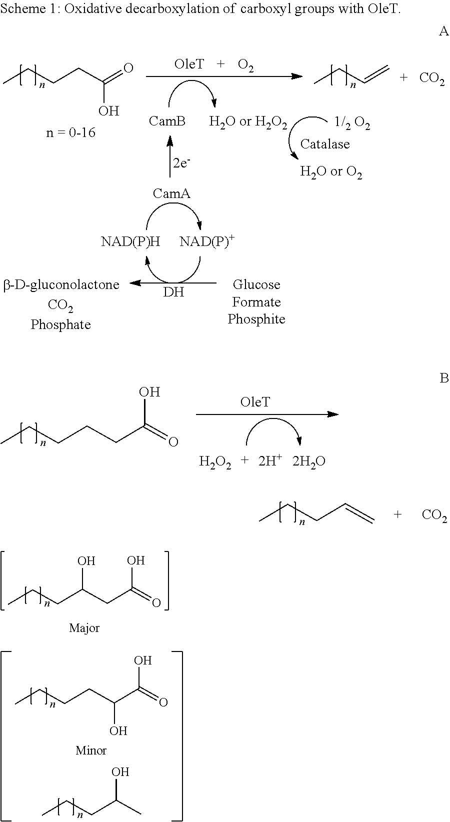

Stringency of hybridisation reactions is readily determinable by one ordinary skilled in the art, and generally is an empirical calculation dependent on probe length, washing temperature and salt concentration. In general, longer probes require higher temperatures for proper annealing, while shorter probes need lower temperatures. Hybridisation generally depends on the ability of denatured DNA to reanneal to complementary strands when present in an environment below their melting temperature. The higher the degree of desired homology between the probe and hybridisable sequence, the higher the relative temperature which may be used. As a result it follows that higher relative temperatures would tend to make the reaction conditions more stringent, while lower temperature less so. For additional details and explanation of stringency of hybridisation reactions, see F. M. Ausubel (1995). The person skilled in the art may follow the instructions given in the manual “The DIG System Users Guide for Filter Hybridization”, Boehringer Mannheim GmbH, Mannheim, Germany, 1993 and in Liebl et al., 1991 on how to identify DNA sequences by means of hybridisation. In one example, stringent conditions are applied for any hybridisation, i.e. hybridisation occurs only if the probe is 70% or more identical to the target sequence. Probes having a lower degree of identity with respect to the target sequence may hybridise, but such hybrids are unstable and will be removed in a washing step under stringent conditions, for example by lowering the concentration of salt to 2×SSC or, optionally and subsequently, to 0.5×SSC, while the temperature is, in order of increasing preference, approximately 50° C.-68° C., approximately 52° C.-68° C., approximately 54° C.-68° C., approximately 56° C.-68° C., approximately 58° C.-68° C., approximately 60° C.-68° C., approximately 62° C.-68° C., approximately 64° C.-68° C., approximately 66° C.-68° C. In a particularly preferred embodiment, the temperature is approximately 64° C.-68° C. or approximately 66° C.-68° C. It is possible to adjust the concentration of salt to 0.2×SSC or even 0.1×SSC. Polynucleotide fragments having a degree of identity with respect to the reference or wild type sequence of at least 70, 80, 90, 91, 92, 93, 94, 95, 96, 97, 98, or 99% may be isolated. The term “homologue” of a nucleic acid sequence, as used herein, refers to any nucleic acid sequence that encodes the same amino acid sequence as the reference nucleic acid sequence, in line with the degeneracy of the genetic code.

A skilled person would be capable of easily measuring the activity of each of the enzymes E₁, E₂ and E₃. For example, to determine if the expression of E₁ is increased in a cell, a skilled person may use the assay disclosed in Liu et al, 2014, Rude M. A, 2011, Schallmey, A., 2011, and the like. For example, to determine if the expression of E₂ is increased in a cell, a skilled person may use the assay disclosed in Scheps, D, 2011, Roome et al., Schallmey et al. and the like. The expression of E₃ in a cell, whether it is increased or decreased, may be measured using the assay disclosed at least in Cartel et al. where formate dehydrogenase activity determination (via NAD(P)+ reduction is determined as change in absorbance at 340 nm. A skilled person would easily be able to identify other well-known methods in the art that may be used for measuring the expression of the enzymes used in the cell of the present invention.

The cell according to any aspect of the present invention may have reduced capacity of fatty acid degradation by beta-oxidation relative to the wild type cell. In particular, the reduced fatty acid degradation activity compared to the wild type cell may be a result of decreased expression relative to the wild type cell of at least one enzyme selected from the group consisting of acyl-CoA dehydrogenase (FadE) (E₆) (EC:1.3.99.-), enoyl-CoA hydratase (FadB) (E₇) (EC 4.2.1.17), (R)-3-hydroxyacyl-CoA dehydrogenase (FadB) (E₈) (EC 1.1.1.35) and 3-ketoacyl-CoA thiolase (FadA) (E₉) (EC:2.3.1.16).

The term “having a reduced fatty acid degradation capacity”, as used herein, means that the respective cell degrades fatty acids, in particular those taken up from the environment, at a lower rate than a comparable cell or wild type cell having normal fatty acid degradation capacity would under identical conditions. In one example, the fatty acid degradation of such a cell is lower on account of deletion, inhibition or inactivation of at least one gene encoding an enzyme involved in the β-oxidation pathway. In one example, at least one enzyme involved in the β-oxidation pathway has lost, in order of increasing preference, 5, 10, 20, 40, 50, 75, 90 or 99% activity relative to the activity of the same enzyme under comparable conditions in the respective wild type microorganism. The person skilled in the art may be familiar with various techniques that may be used to delete a gene encoding an enzyme or reduce the activity of such an enzyme in a cell, for example by exposition of cells to radioactivity followed by accumulation or screening of the resulting mutants, site-directed introduction of point mutations or knock out of a chromosomally integrated gene encoding for an active enzyme, as described in Sambrook/Fritsch/Maniatis (1989). In addition, the transcriptional repressor FadR may be over expressed to the effect that expression of enzymes involved in the β-oxidation pathway is repressed (Fujita, Y., et al, 2007). The phrase “deletion of a gene”, as used herein, means that the nucleic acid sequence encoding said gene is modified such that the expression of active polypeptide encoded by said gene is reduced. For example, the gene may be deleted by removing in-frame a part of the sequence comprising the sequence encoding for the catalytic active centre of the polypeptide. Alternatively, the ribosome binding site may be altered such that the ribosomes no longer translate the corresponding RNA. It would be within the routine skills of the person skilled in the art to measure the activity of enzymes expressed by living cells using standard essays as described in enzymology text books, for example Cornish-Bowden, 1995.

Degradation of fatty acids is accomplished by a sequence of enzymatically catalysed reactions. First of all, fatty acids are taken up and translocated across the cell membrane via a transport/acyl-activation mechanism involving at least one outer membrane protein and one inner membrane-associated protein which has fatty acid-CoA ligase activity, referred to in the case of E. coli as FadL and FadD/FadK, respectively. Inside the cell, the fatty acid to be degraded is subjected to enzymes catalysing other reactions of the β-oxidation pathway. The first intracellular step involves the conversion of acyl-CoA to enoyl-CoA through acyl-CoA dehydrogenase, the latter referred to as FadE in the case of E. coli. The activity of an acyl-CoA dehydrogenase may be assayed as described in the state of art, for example by monitoring the concentration of NADH spectrophotometrically at 340 nm in 100 mM MOPS, pH 7.4, 0.2 mM Enoyl-CoA, 0.4 mM NAD⁺. The resulting enoyl-CoA is converted to 3-ketoacyl-CoA via 3-hydroxylacyl-CoA through hydration and oxidation, catalysed by enoyl-CoA hydratase/(R)-3-hydroxyacyl-CoA dehydrogenase, referred to as FadB and FadJ in E. coli. Enoyl-CoA hydratase/3-hydroxyacyl-CoA dehydrogenase activity, more specifically formation of the product NADH may be assayed spectrophotometrically as described in the state of the art, for example as outlined for FadE. Finally, 3-ketoacyl-CoA thiolase, FadA and FadI in E. coli, catalyses the cleavage of 3-ketoacyl-CoA, to give acetyl-CoA and the input acyl-CoA shortened by two carbon atoms. The activity of ketoacyl-CoA thiolase may be assayed as described in the state of the art, for example in Antonenkov, V., et al, 1997.

The phrase “a cell having a reduced fatty acid degradation capacity”, as used herein, refers to a cell having a reduced capability of taking up and/or degrading fatty acids, particularly those having at least eight carbon chains. The fatty acid degradation capacity of a cell may be reduced in various ways. In particular, the cell according to any aspect of the present invention has, compared to its wild type, a reduced activity of an enzyme involved in the β-oxidation pathway. The term “enzyme involved in the β-oxidation pathway”, as used herein, refers to an enzyme that interacts directly with a fatty acid or a derivative thereof formed as part of the degradation of the fatty acid via the β-oxidation pathway. The β-oxidation pathway comprises a sequence of reactions effecting the conversion of a fatty acid to acetyl-CoA and the CoA ester of the shortened fatty acid. The enzyme involved in the β-oxidation pathway may by recognizing the fatty acid or derivative thereof as a substrate, converts it to a metabolite formed as a part of the β-oxidation pathway. For example, the acyl-CoA dehydrogenase (EC 1.3.99.-) is an enzyme involved in the β-oxidation pathway as it interacts with fatty acid-CoA and converts fatty acid-CoA ester to enoyl-CoA, which is a metabolite formed as part of the β-oxidation. In another example, the term “enzyme involved in the β-oxidation pathway”, as used herein, comprises any polypeptide from the group comprising acyl-CoA dehydrogenase (EC 1.3.99.-), enoyl-CoA hydratase (EC 4.2.1.17), 3-hydroxyacyl-CoA dehydrogenase EC 1.1.1.35) and 3-keto-acyl-CoA thiolase (EC 2.3.1.16). The acyl-CoA synthetase (EC 6.2.1.1) may catalyse the conversion of a fatty acid to the CoA ester of a fatty acid, i.e. a molecule, wherein the functional group —OH of the carboxy group is replaced with —S-CoA and introducing the fatty acid into the β-oxidation pathway. For example, the polypeptides FadD and FadK in E. coli (accession number: BAA15609.1 and NP_416216.4, respectively) are acyl-CoA dehydrogenases. In one example, the term “acyl-CoA dehydrogenase”, as used herein, may be a polypeptide capable of catalysing the conversion of an acyl-CoA to enoyl-CoA, as part of the β-oxidation pathway. For example, the polypeptide FadE in E. coli (accession number: BAA77891.2) may be an acyl-CoA dehydrogenase. The term “enoyl-CoA hydratase”, as used herein, also referred to as 3-hydroxyacyl-CoA dehydrogenase, refers to a polypeptide capable of catalysing the conversion of enoyl-CoA to 3-ketoacyl-CoA through hydration and oxidation, as part of the β-oxidation pathway. For example, the polypeptides FadB and FadJ in E. coli (accession numbers: BAE77457.1 and P77399.1, respectively) are enoyl-CoA hydratases. The term “ketoacyl-CoA thiolase”, as used herein, may refer to a polypeptide capable of catalysing the cleaving of 3-ketoacyl-CoA, resulting in an acyl-CoA shortened by two carbon atoms and acetyl-CoA, as the final step of the β-oxidation pathway. For example, the polypeptides FadA and FadI in E. coli (accession number: YP_491599.1and P76503.1, respectively) are ketoacyl-CoA thiolases.

The term “contacting”, as used herein, means bringing about direct contact between the amino acid used as a substrate, and the cell according to any aspect of the present invention in an aqueous solution. For example, the cell and the amino acid may be in different compartments separated by a barrier such as an inorganic membrane. If the amino acid is soluble and may be taken up by the cell or can diffuse across biological membranes, it may simply be added to the cell according to any aspect of the present invention in an aqueous solution. In case it is insufficiently soluble, it may be dissolved in a suitable organic solvent prior to addition to the aqueous solution. The person skilled in the art is able to prepare aqueous solutions of amino acids having insufficient solubility by adding suitable organic and/or polar solvents. Such solvents may be provided in the form of an organic phase comprising liquid organic solvent. In one example, the organic solvent or phase may be considered liquid when liquid at 25° C. and standard atmospheric pressure. In another example, the compounds and catalysts may be contacted in vitro, i.e. in a more or less enriched or even purified state, or may be contacted in situ, i.e. they are made as part of the metabolism of the cell and subsequently react inside the cell.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep the cells used in the method according to any aspect of the present invention, for example LB medium in the case of E. coli. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium.

According to any aspect of the present invention, the amino acid comprising at least two carbonyl groups may be added to an aqueous solution comprising the cell according to any aspect of the present invention. This step may not only comprise temporarily contacting the amino acid with the solution, but in fact incubating the amino acid in the presence of the cell sufficiently long to allow for an oxidation reaction and possible further downstream reactions to occur, for example for at least 1, 2, 4, 5, 10 or 20 hours. The temperature chosen must be such that the cells according to any aspect of the present invention remains catalytically competent and/or metabolically active, for example 10 to 42° C., in particular 30 to 40° C., more in particular, 32 to 38° C. in case the cell is an E. coli cell.

In particular, the cofactor of the method according to any aspect of the present invention may be NAD+/NADH. More in particular, the method further comprises a coupled process of cofactor regeneration for regenerating the consumed cofactor NAD(P)+. The coupled cofactor regenerating process also comprises the regeneration of the consumed sacrificial glucose, formate, phosphine or the like.

In one example, the unsaturated amino acid formed according to any aspect of the present invention may be vinylglycine and derivatives thereof. In this example, the method according to any aspect of the present invention may comprise a further step of

(b) contacting the vinylglycine or derivatives thereof with a free radical methyl mercaptan. This is a step that results in the formation of methionine. In particular, this step is part of the chemical process of making methionine. Methyl mercaptan also known as methanethiol has a chemical formula of CH₄S and structure of Formula II:

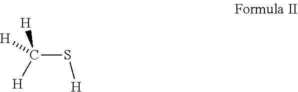

The free-radical addition of a methyl mercaptan to vinylglycine may result in the radicalized methyl mercaptan to acting on the terminal carbon-carbon double bond of vinylglycine to produce 2-amino 4-(methylthio) butanoic acid. This step has an advantage of producing L- and/or D-methionine economically through having high conversion rates and short reaction time. Further, compared to methods used in the art where acetylhomoserine is used as the substrate for methyl mercaptan activity, the use of vinylglycine has other advantages. For example, using acetylhomoserine as the substrate for methyl mercaptan activity results in the production of a side product, acetic acid. This production may be considered to be a loss in carbon, where not all the carbon from the substrate (i.e. acetylhomoserine) is converted to the target product, methionine. Also, with acetic acid release, the methionine partly absorbs the scent of acetate. The methionine produced using this method thus has a trace of acetate. These problems may be overcome by the method according to any aspect of the present invention. The method according to any aspect of the present invention thus has an advantage of producing L-methionine and/or D-methionine economically through having high conversion rates and short reaction time.

On the other hand, using vinylglycine as a substrate for the activity of radicalized methyl mercaptan does not have the same disadvantages as those mentioned when acetylhomoserine is used. Firstly, there is no loss of carbon as all the carbon in vinylglycine is converted to be part of methionine. There is also no production of acetic acid. Further, the substrate vinylglycine can be synthesized easily from readily available glutamate, the amino acid with one of the highest production volumes in living things. The glutamate may be the L and/or the D isomer. The radicalized methyl mercaptan step, also known as Thiol-ene coupling reaction, may also be considered to be relatively selective as no side product may be released when vinylglycine is used as the substrate.

The free radicalization of methyl mercaptan by any means known in the art may result in the breaking of the sulfur-hydrogen bond in methyl mercaptan to produce a methyl mercaptan free radical.

The methyl mercaptan free radical may then act across the terminal carbon-carbon double bond in the vinylglycine. This action may result in the double bond being reduced to a single bond and a methylthio group added according to the Anti-Markovnikov rule at the terminal carbon atom. The unpaired electron on the adjacent, non-terminal carbon atom in the substrate binds with a hydrogen atom supplied by the methyl mercaptan, thereby creating another methyl mercaptan free radical and this continues the addition cycle.

In particular, the ratio of methyl mercaptan to vinylglycine or derivatives thereof may be 1:1, particularly in the reaction medium. However, a skilled person would be capable of varying this ratio depending on the initiator used to form the radical. In one example, the ratio of methyl mercaptan to vinylglycine or derivatives thereof may be selected from the range of 1:1 to 1:10. In particular, the ratio may be 1.2:1. In one example, the ratio of methyl mercaptan to vinylglycine or derivatives thereof may be selected from 3:1-6:1. This may be advantageous according to any aspect of the present invention as in Thiol-ene coupling reactions, an excess of Thiol may be necessary.

In one example, the free radicalization of methyl mercaptan may be carried out by contacting the methyl mercaptan with at least one free radical initiator. There are several initiators that may be used according to any aspect of the present invention. A skilled person may be capable of identifying these initiators. For example, the free radical initiator may be selected from the group consisting of azobisisobutyronitrile (AIBN), N-bromosuccinimide (NBS), dibenzoyl peroxide (DBPO), Vazo®-44 (2,2′-azobis[2-(2-imidazolin-2-yl)propane]dichloride) and the like. When in contact with any of these free radical initiators, the methyl mercaptan may be radicalized to produce a free radical that may then react with the vinylglycine to produce methionine. In one example, AIBN is the free radical initiator. AIBN is thermally stable at room temperature. However, upon being heated to an activation temperature it produces a free radical which may then start the free radical addition chain reaction with vinylglycine. In another example, the Vazo®-44 may be the free radical initiator. The VAZO® series of free radical initiators are available from DuPont Chemicals of Wilmington, Del., U.S.A. In particular, the free radical initiator may be selected from the group consisting of azobisisobutyronitrile (AIBN) and 2,2-azobis (2-(2-imidazolin-2-yl)propane) dihydrochloride.

In another example, instead of using a chemical agent like a free radical initiator to radicalize methyl mercaptan, an ultraviolet light source may be used. The UV light may be at wavelengths of 300 nm or 365 nm. In particular, the UV light may have a wavelength of 300 nm.

In a further example, free radicalization of the methyl mercaptan may be carried out by a combination of UV light and a photo initiator such as 2,2-Dimethoxy-2-phenylacetophenone (DPAP). In this example, the UV light may have a wavelength of 365 nm.

In one example, free radicalization of the methyl mercaptan may be carried out without an additional initiator. In this example, no chemical initiator and/or UV rays are needed. Radicalization of methyl mercaptan may take place autocatalytically upon heating or may assisted by ultrasonic sound or impurities (e.q. oxygen). A skilled person would be capable of carrying out the radicalization using a variety of means. Reactions without additional chemical initiator may however suffer from low reaction rates and yields.

In all the above examples, the step of free radicalization of methyl mercaptan may be carried out at the same time as the conversion of vinylglycine to methionine. Therefore, both steps of free radicalization and conversion of vinylglycine to methionine may be carried out in the same pot. For example, when a temperature activated free radical initiator such as AIBN is used, the temperature and pressure conditions of the reaction are firstly maintained such that the reactants (i.e. methyl mercaptan, vinylglycine and AIBN) are present as liquids and the temperature is below the activation temperature of the free radical initiator. The order of introduction of the reactants and free radical initiator into the pot is unimportant as the conditions of the reaction mixture in the pot are such that essentially no reaction occurs. When the temperature is increased, the reaction kick starts and radicalized AIBN results in the formation of the free radical of methyl mercaptan which then attacks the C double bond in vinylglycine to form methionine.

In particular, the ratio of free radical initiator to methyl mercaptan may be within the range of 1:10000 to 1:5. More in particular, the ratio of the free radical initiator to methyl mercaptan may be within the range of 1:10000 to 1:10. Even more in particular, the ratio of the free radical initiator to methyl mercaptan may be about 1:1000, 1:500, 1:100, 1:50, 1:20, 1:30, 1:10, 1:3 and the like.

In another example, the pot may have a translucent portion (e.g., a reactor window) where UV light may be shone into the pot. Alternatively, the ultraviolet light source may be disposed within a translucent envelope extending into the pot. The UV light in the reaction pot may then radicalize the methyl mercaptan in the pot. The process may take at least about 5 hours or more. The reaction mixture may then be cooled to room temperature and excess methyl mercaptan may be allowed to volatilize and is removed from the reaction pot. The excess methyl mercaptan may then be recovered for reuse. Methionine may then be left behind in the pot.

In a further example, the pot with a translucent portion may comprise vinylglycine, a photo initiator like DPAP and methyl mercaptan. Without UV light, no reaction takes place in the pot. When UV light at 365 nm is introduced into the pot by any means known in the art, the photo initiator may be activated to radicalize methyl mercaptan. The free radical of methyl mercaptan may then act on vinylglycine to produce methionine. The excess vinylglycine may then be removed as described above and recycled. The resultant product in the pot may then be only methionine.

According to another aspect of the present invention, there is provided a method of producing methionine, the method comprising,
(a) contacting a recombinant microbial cell with a medium comprising glutamic acid to produce vinylglycine and/or derivatives thereof,
(b) contacting the vinylglycine or derivatives thereof of (a) with a free radical methylmercaptan,
wherein the cell is genetically modified to comprise
   at least a first genetic mutation that increases the expression relative to the wild type cell of an enzyme (E₁) selected from the CYP152 peroxygenase family, and
   at least a second genetic mutation that increases the expression relative to the wild type cell of at least one NAD(P)+ oxidoreductase (E₂) and the corresponding mediator protein.

The method of producing methionine according to any aspect of the present invention may be a two pot process. In one pot, step (a) may be carried out where the cell according to any aspect of the present invention contacts an aqueous medium comprising glutamic acid. The conditions in pot one are maintained to optimize production of vinylglycine. A skilled person would be capable of identifying the suitable conditions for optimized activity of the cells in this pot to produce vinylglycine. The vinylglycine may then be concentrated or separated by any means known in the art from pot 1. In one example, vinylglycine may be separated from the solution of pot 1 by precipitation or extraction and the resultant vinylglycine transferred into a second pot, pot 2. In another example, all the contents of pot 1 are transferred to pot 2. Pot 1 may constantly be refilled with glutamic acid and the cells recycled to keep the cost low. In another example, vinylglycine formed is allowed to accumulate in pot one before vinylglycine is extracted and transferred to pot two. In this example, pot two, before the introduction of vinylglycine may already comprise (i) a temperature activated free radical initiator such as AIBN and methyl mercaptan. When vinylglycine may be introduced into pot 2, the temperature and pressure conditions of pot 2 are firstly maintained such that the reactants (i.e. methyl mercaptan, vinylglycine and AIBN) are present as liquids and the temperature is below the activation temperature of the free radical initiator. When the temperature is increased, the reaction kick starts and radicalized AIBN results in the formation of the free radical of methyl mercaptan which then attacks the C double bond in vinylglycine to form methionine in pot 2.

In another example, vinylglycine from pot 1 may be introduced into pot 2 that comprises methyl mercaptan and which may have a translucent portion (e.g., a reactor window) where UV light may be shone into the pot. Alternatively, the ultraviolet light source may be disposed within a translucent envelope extending into the pot. The UV light introduced into pot 2 may then radicalize the methyl mercaptan in the pot. The process may take at least about 5 hours or more. The reaction mixture may then be cooled to room temperature and excess methyl mercaptan may be allowed to volatilize and is removed from the reaction pot. The excess methyl mercaptan may then be recovered for reuse. Methionine may then be left behind in the pot 2.

In a further example, vinylglycine from pot 1 may be introduced into pot 2 that comprises methyl mercaptan, photo initiator like DPAP and a translucent portion. Without UV light, no reaction takes place in the pot. When UV light at 365 nm is introduced into the pot by any means known in the art, the photo initiator may be activated to radicalize methyl mercaptan. The free radical of methyl mercaptan may then act on vinylglycine to produce methionine. The excess vinylglycine may then be removed as described above and recycled. The resultant product in the pot 2 may then be only methionine.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Synthesis of Methionine Starting from Vinylglycine via Thiol-Ene-Coupling (TEC)

In a flask (250 mL) is equipped with a reflux condenser vinylglycine (1.011 g, 10.00 mmol, 1.00 eq.) is dissolved in Methanol/Water (1/1, 40 mL) and AIBN (0.164 g, 1.00 mmol, 0.10 eq.) is added. Methyl mercaptan (2.887 g, 2.60 mL, 60.00 mmol, 6.00 eq.) is condensed at −30° C. in a second flask acting as a reservoir. The cooling bath is removed and the reservoir connected to the reaction apparatus to pass the methyl mercaptan through the reaction mixture, while the mixture is heated at 60° C. for 6 hours. The reaction is cooled down to ambient temperature and the formed precipitate collected by filtration to obtain the title compound (as a white crystalline solid of methionine). The structural integrity of the product is confirmed by NMR.

### Example 2

Vinylglycine was Formed Using OleT by Oxidative Decarboxylation of Glutamate in an Aqueous Solution.

### Example 3

Production of N-Acetylvinylglycine from N-Acetylglutamate with OleT

For the biotransformation of N-acetylglutamate to N-acetylvinylglycine a biocatalytic system with purified enzymes of a P450 monooxygenase (OleT), an electron-transfer system (CamAB) and a formiat dehydrogenase (FDH) were used in the presence of formate, oxygen and NADH.

All chemicals were obtained from Sigma Aldrich (Steinheim, Germany) unless otherwise stated; N-acetyl glutamic acid was obtained from Alfa Aesar (Thermo Fisher, Karlsruhe, Germany), ammonium formate from Carl Roth (Karlsruhe, Germany), NADH-disodium salt from Panreac (Barcelona, Spain).

Catalase from bovine liver, lysozyme from chicken egg and cytochrome c from bovine heart were obtained from Sigma Aldrich (Steinheim, Germany), formate dehydrogenase (NADH-dependent) was obtained from Evocatal (Monheim am Rhein, Germany). The plasmid for expression of CamAB was obtained from Anett Schallmey (TU Braunschweig, Germany). Expression and purification of OleT, as well as expression and activity determination of CamAB electron transfer system, were run according to a standard protocol developed by Dennig et al, Angew. Chem. Int. Ed. 2015, 54, 8819.

For the biotransformation 6 μM OleT, 10 mM N-acetylglutamate, 0.05 U/mL CamAB, 1200 U/mL catalase, 2 U/mL FDH, 100 mM NH₄COOH and 200 μM NADH in phosphate buffer (100 mM, pH 7.5) were shaken at room temperature in a 4 mL glass vial at 160 rpm for 24 hours. For a control reaction the same conditions were used, except that OleT was omitted. All the reactions were performed in duplicates.

For derivatization of the product, an aliquot (900 μL) of the sample was transferred into a 1.5 mL glass vial, treated with 150 μL of a solution of NaIO₄ (10 mM) and shaken at 25° C. at 1000 rpm for 30 minutes. Water was then removed by a centrifugal evaporator (SpeedVac), and the residue was dissolved by vortexing in 700 μL of a solution of MeOH containing 5% DMAP (4-dimethylaminopyridine); then 150 μL of ethylchloroformate were added and the mixture was heated for 1 hour at 50° C. with shaking at 800 rpm. Solvent was then removed by SpeedVac, the residue was dissolved in 700 μL of aq. 2% HCl and extracted twice with 200 μL of EtOAc (spiked with 5 mM (R)-limonene as internal standard); the collected organic fractions were dried over Na₂SO₄.

The samples were analyzed as derivatized amino acids on an Agilent 7890A GC (Gas chromatography) system (H₂ as carrier gas) equipped with an FID (flame ionization detector), using an Agilent DB-1701 column (30 m×350 μm, 0.25 μm film); injection volume: 5 μL, split ratio: 50:1, injection temperature: 250° C., detection temperature: 250° C.; program: 100° C./hold 3 min, 20° C./min to 280° C., hold 1 min.

The analysis of the sample of the biotransformation with OleT revealed the presence of a small peak, which had the same retention time (Rt=6.2 min) of the carbamate-derivatized form of vinylglycine (the reference compound was synthesized independently). The co-injection of a small amount of the synthesized reference material led to an increase of the area of the new peak.

1. A method of producing at least one unsaturated amino acid from at least one amino acid comprising at least two carbonyl groups, the method comprising
(a) contacting a recombinant microbial cell with a medium comprising the amino acid comprising the carbonyl groups,
wherein the cell is genetically modified to comprise
at least a first genetic mutation that increases the expression relative to the wild type cell of an enzyme (E₁) selected from the CYP152 peroxygenase family, and
at least a second genetic mutation that increases the expression relative to the wild type cell of at least one NAD(P)+ oxidoreductase (E₂) and the corresponding mediator protein.

2. The method according to claim 1, wherein the amino acid comprising at least two carbonyl groups is selected from the group consisting of aspartic acid, glutamic acid, asparagine and glutamine.

3. The method according to claim 1, wherein the unsaturated amino acid is vinylglycine or derivatives thereof,

4. The method according to claim 1, wherein E₁ is selected from the group consisting of CYP_{SPα} (E₁ₐ) CYP_{BSB} (E_{1b}) and OleT (E_{1c}).

5. The method according to claim 1, wherein E₁ is OleT (E_{1c}) and comprises at least 60% sequence identity to SEQ ID NO:1.

6. The method according to claim 1, wherein the NAD(P)+ oxidoreductase (E₂) and the corresponding mediator protein are selected from the group consisting of:
ferredoxin reductase (E₂ₐ) and ferredoxin; and
putidaredoxin reductase (E_{2b}) and putidaredoxin.

7. The method according to claim 1, wherein E₂ comprises 60% sequence identity to SEQ ID NO:2 and the mediator protein comprises 60% sequence identity to SEQ ID NO:3.

8. The method according to claim 1, wherein the cell further comprises at least a third genetic mutation that increases the expression relative to the wild type cell of at least one enzyme (E₃) capable of NAD(P)H regeneration.

9. The method according to claim 8, wherein the enzyme (E₃) is selected from the group consisting of glucose dehydrogenase, phosphite dehydrogenase and formate dehydrogenase.

10. The method according to claim 1, wherein the cell further comprises a reduced fatty acid degradation capacity relative to the wild type cell.

11. The method according to claim 10, wherein the fatty acid degradation capacity is reduced by deletion of a gene encoding an enzyme selected from the group consisting of fatty acid importer, fatty acid-CoA ligase, acyl-CoA dehydrogenase, 2,4-dienoyl-CoA reductase, enoyl-CoA hydratase and 3-ketoacyl-CoA thiolase.

12. The method according to claim 3, further comprises a step of
(b) contacting the vinylglycine or derivatives thereof with a free radical methyl mercaptan.

13. A method of producing methionine, the method comprising,
(a) contacting a recombinant microbial cell with a medium comprising glutamic acid to produce vinylglycine and/or derivatives thereof,
(b) contacting the vinylglycine or derivatives thereof of (a) with a free radical methyl mercaptan,
wherein the cell is genetically modified to comprise
at least a first genetic mutation that increases the expression relative to the wild type cell of an enzyme (E₁) selected from the CYP152 peroxygenase family, and
at least a second genetic mutation that increases the expression relative to the wild type cell of at least one NAD(P)+ oxidoreductase (E₂) and the corresponding mediator protein.

14. The method according to claim 13, wherein E₁ is OleT (E_{1c}) and comprises at least 60% sequence identity to SEQ ID NO:1.

15. The method according to claim 13, wherein the NAD(P)+ oxidoreductase (E₂) and the corresponding mediator protein are selected from the group consisting of:
ferredoxin reductase (E₂ₐ) and ferredoxin; and
putidaredoxin reductase (E_{2b}) and putidaredoxin.
